(19) United States
(12) Patent Application Publication
(10) Pub. No.: US 2004/0029245 A1
(43) Pub. Date: Feb. 12, 2004

(54) Regulation of human serine-palmitoyltransferase-like enzyme

(76) Inventor: Sophia Kossida, Basel (CH)

Correspondence Address: BANNER & WITCOFF, 1001 G STREET N W, SUITE 1100, WASHINGTON, DC, 20001, US

(21) Appl. No.: 10/380,173

(22) PCT Filed: Sep. 17, 2001
(86) PCT No.: PCT/EP01/10697

(51) Int. Cl. C12N 9/10; C07H 21/04; C12P 21/02; C12N 5/06

(52) U.S. Cl. 435/193; 435/69.1; 435/320.1; 435/325; 536/23.2

(57) ABSTRACT

Reagents which regulate human serine palmitoyltransferase-like enzyme and reagents which bind to human serine palmitoyltransferase-like enzyme gene products can play a role in preventing, ameliorating, or correcting dysfunctions or diseases including, but not limited to, cancer, diabetes, and obesity.

### TECHNICAL FIELD OF THE INVENTION

The invention relates to the area of enzyme regulation. More particularly, the invention relates to the regulation of human serine palmitoyltransferase-like enzyme and its regulation.

### BACKGROUND OF THE INVENTION

Serine palmitoyltransferase (EC 2.3.1.50) catalyzes the formation of a homologue of 3-ketosphinganine from L-serine and a fatty acyl coenzyme A thioester, which is the initial reaction of sphingolipid biosynthesis. Williams et al., Cancer Res. 44, 1918-23, 1984. It has recently been proposed that serine palmitoyltransferase is the enzyme which controls de novo ceramide synthesis during apoptosis. Perry et al., J. Biol. Chem. 275, 9078-84, 2000.

Serine palmitoyltransferase has been implicated in cancer. See, e.g., Williams et al., 1984; Schroeder et al., J. Biol. Chem. 269, 3475-81, 1994. Serine palmitoyltransferase activity also has been implicated in the lipoapoptosis of obese diabetic rats. Shimabukuro et al., J. Biol. Chem. 273, 32487-90,-1998. There is, therefore, a need in the art to identify related enzymes which can be regulated to provide therapeutic effects.

### SUMMARY OF THE INVENTION

It is an object of the invention to provide reagents and methods of regulating a human serine palmitoyltransferase-like enzyme. This and other objects of the invention are provided by one or more of the embodiments described below.

One embodiment of the invention is a serine palmitoyltransferase-like enzyme polypeptide comprising an amino acid sequence selected from the group consisting of:

amino acid sequences which are at least about 50% identical to the amino acid sequence shown in SEQ ID NO: 2,

the amino acid sequence shown in SEQ ID NO: 2;

amino acid sequences which are at least about 50% identical to the amino acid sequence shown in SEQ ID NO: 7, and

the amino acid sequence shown in SEQ ID NO: 7.

Yet another embodiment of the invention is a method of screening for agents which decrease extracellular matrix degradation. A test compound is contacted with a serine palmitoyltransferase-like enzyme polypeptide comprising an amino acid sequence selected from the group consisting of:

amino acid sequences which are at least about 50% identical to the amino acid sequence shown in SEQ ID NO: 2,

the amino acid sequence shown in SEQ ID NO: 2;

amino acid sequences which are at least about 50% identical to the amino acid sequence shown in SEQ ID NO: 7, and

the amino acid sequence shown in SEQ ID NO: 7.

Binding between the test compound and the serine palmitoyltransferase-like enzyme polypeptide is detected. A test compound which binds to the serine palmitoyltransferase-like enzyme polypeptide is thereby identified as a potential agent for decreasing extracellular matrix degradation. The agent can work by decreasing the activity of the serine palmitoyltransferase-like enzyme.

Another embodiment of the invention is a method of screening for agents which decrease extracellular matrix degradation. A test compound is contacted with a polynucleotide encoding a serine palmitoyltransferase-like enzyme polypeptide, wherein the polynucleotide comprises a nucleotide sequence selected from the group consisting of:

nucleotide sequences which are at least about 50% identical to the nucleotide sequence shown in SEQ ID NO: 1,

the nucleotide sequence shown in SEQ ID NO: 1;

nucleotide sequences which are at least about 50% identical to the nucleotide sequence shown in SEQ ID NO: 6,

the nucleotide sequence shown in SEQ ID NO: 6.

Binding of the test compound to the polynucleotide is detected. A test compound which binds to the polynucleotide is identified as a potential agent for decreasing extracellular matrix degradation. The agent can work by decreasing the amount of the serine palmitoyltransferase-like enzyme through interacting with the serine palmitoyltransferase-like enzyme mRNA.

Another embodiment of the invention is a method of screening for agents which regulate extracellular matrix degradation. A test compound is contacted with a serine palmitoyltransferase-like enzyme polypeptide comprising an amino acid sequence selected from the group consisting of:

amino acid sequences which are at least about 50% identical to the amino acid sequence shown in SEQ ID NO: 2, and

the amino acid sequence shown in SEQ ID NO: 2;

amino acid sequences which are at least about 50% identical to the amino acid sequence shown in SEQ ID NO: 7, and

the amino acid sequence shown in SEQ ID NO: 7.

A serine palmitoyltransferase-like enzyme activity of the polypeptide is detected. A test compound which increases serine palmitoyltransferase-like enzyme activity of the polypeptide relative to serine palmitoyltransferase-like enzyme activity in the absence of the test compound is thereby identified as a potential agent for increasing extracellular matrix degradation. A test compound which decreases serine palmitoyltransferase-like enzyme activity of the polypeptide relative to serine palmitoyltransferase-like enzyme activity in the absence of the test compound is thereby identified as a potential agent for decreasing extracellular matrix degradation.

Even another embodiment of the invention is a method of screening for agents which decrease extracellular matrix degradation. A test compound is contacted with a serine palmitoyltransferase-like enzyme product of a polynucleotide which comprises a nucleotide sequence selected from the group consisting of:

nucleotide sequences which are at least about 50% identical to the nucleotide sequence shown in SEQ ID NO: 1,

the nucleotide sequence shown in SEQ ID NO: 1;

nucleotide sequences which are at least about 50% identical to the nucleotide sequence shown in SEQ ID NO: 6,

the nucleotide sequence shown in SEQ ID NO: 6.

Binding of the test compound to the serine palmitoyltransferase-like enzyme product is detected. A test compound which binds to the serine palmitoyltransferase-like enzyme product is thereby identified as a potential agent for decreasing extracellular matrix degradation.

Still another embodiment of the invention is a method of reducing extracellular matrix degradation. A cell is contacted with a reagent which specifically binds to a polynucleotide encoding a serine palmitoyltransferase-like enzyme polypeptide or the product encoded by the polynucleotide, wherein the polynucleotide comprises a nucleotide sequence selected from the group consisting of:

nucleotide sequences which are at least about 50% identical to the nucleotide sequence shown in SEQ ID NO: 1,

the nucleotide sequence shown in SEQ ID NO: 1;

nucleotide sequences which are at least about 50% identical to the nucleotide sequence shown in SEQ ID NO: 6, and

the nucleotide sequence shown in SEQ ID NO: 6.

Serine palmitoyltransferase-like enzyme activity in the cell is thereby decreased.

The invention thus provides a human serine palmitoyltransferase-like enzyme which can be used to identify test compounds which may act, for example, as agonists or antagonists at the enzyme's active site. Human serine palmitoyltransferase-like enzyme and fragments thereof also are useful in raising specific antibodies which can block the enzyme and effectively reduce its activity.

### BRIEF DESCRIPTION OF THE DRAWINGS

FIG. 1 shows the DNA-sequence encoding a serine palmitoyltransferase-like enzyme polypeptide (SEQ ID NO:1).

FIG. 2 shows the amino acid sequence deduced from the DNA-sequence of FIG. 1 (SEQ ID NO: 2).

FIG. 3 shows the amino acid sequence of the protein identified by SwissProt Accession No. 015270 (SEQ ID NO: 3).

FIG. 4 shows the DNA-sequence encoding a serine palmitoyltransferase-like enzyme polypeptide (SEQ ID NO: 4).

FIG. 5 shows the DNA-sequence encoding a serine palmitoyltransferase-like enzyme polypeptide (SEQ ID NO: 5).

FIG. 6 shows the DNA-sequence encoding a serine palmitoyltransferase-like enzyme polypeptide (SEQ ID NO: 6).

FIG. 7 shows the amino acid sequence deduced from the DNA-sequence of FIG. 6 (SEQ ID NO: 7).

FIG. 8 shows the BLASTP alignment of human serine palmitoyltransferase-like enzyme (SEQ ID NO: 2) with the protein identified with SwissProt Accession No. 015270 (SEQ ID NO: 3).

FIG. 9 shows the FASTA alignment of SEQ ID NO:2 with SEQ ID NO: 3.

FIG. 10 shows the ASTP-alignment of genewise_proteine_{—}223 against swiss/051270/LCB2_HUMAN

FIG. 11 shows the BLASTP-alignment of genewise_proteine 223 against pdb/1BSO/1BSO-A

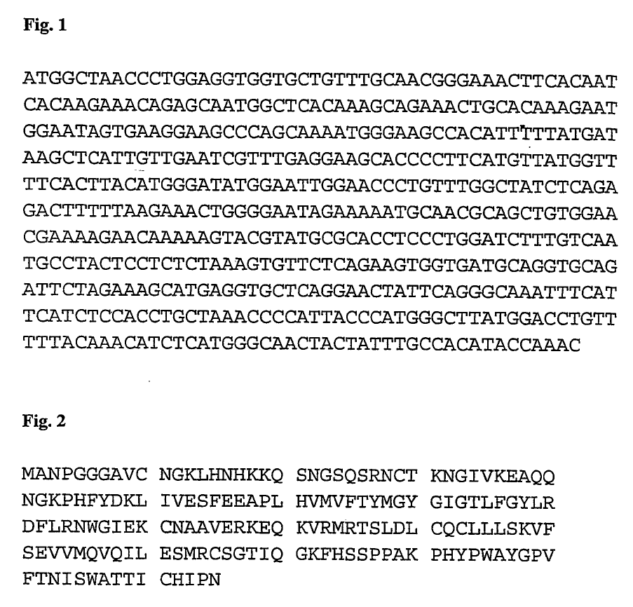

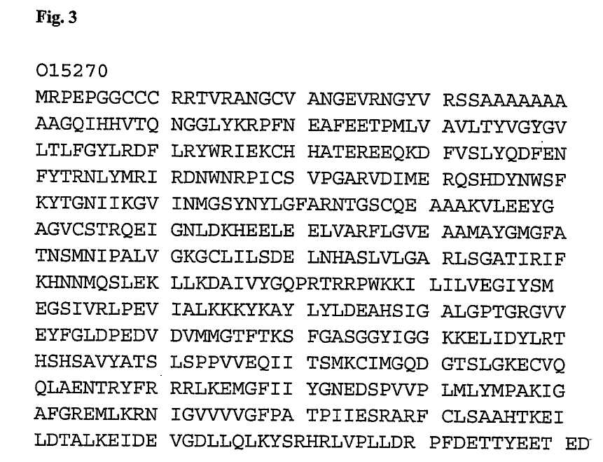

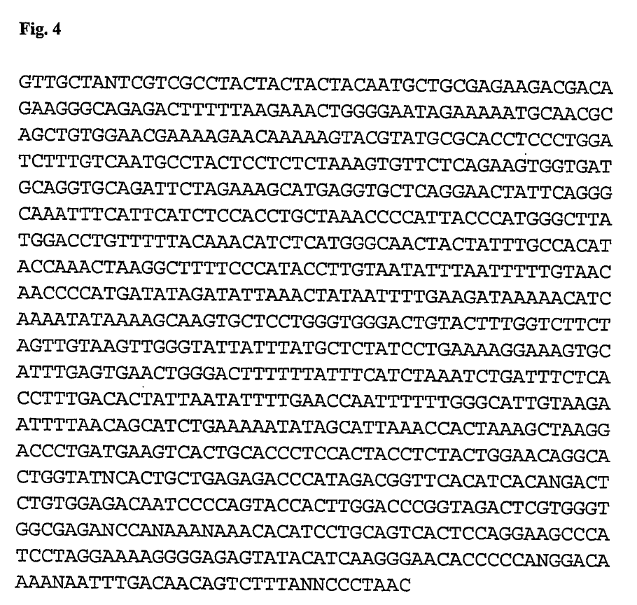

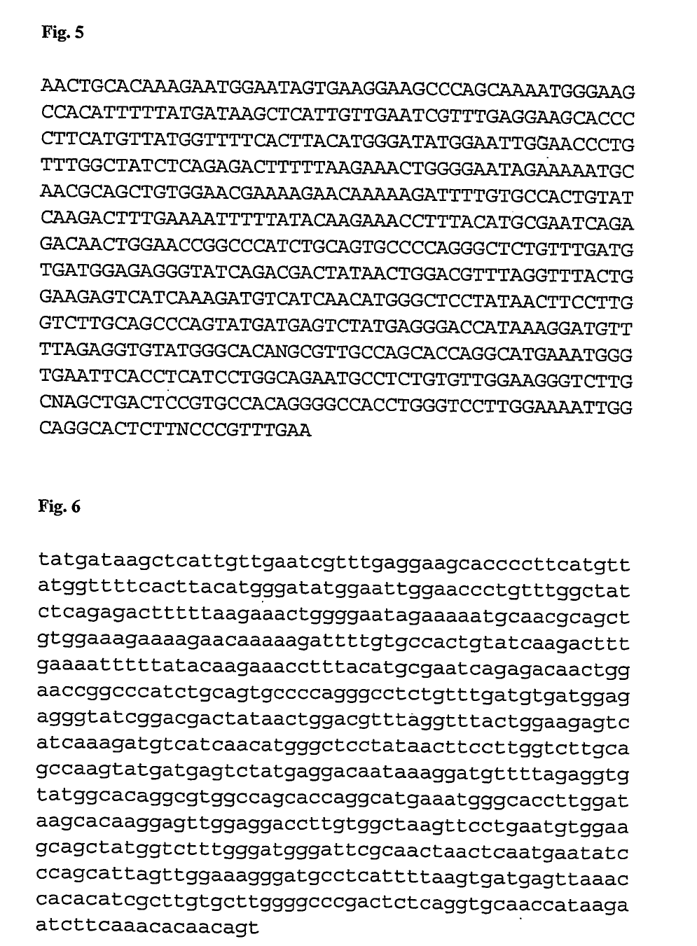

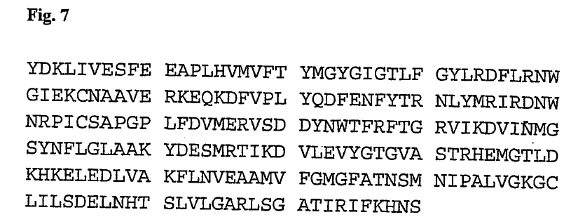

### DETAILED DESCRIPTION OF THE INVENTION

The invention relates to an isolated polynucleotide encoding a serine palmitoyltransferase-like enzyme polypeptide and being selected from the group consisting of:

a) a polynucleotide encoding a serine palmitoyltransferase-like enzyme polypeptide comprising an amino acid sequence selected from the group consisting of:

amino acid sequences which are at least about 50% identical to

the amino acid sequence shown in SEQ ID NO: 2,

the amino acid sequence shown in SEQ ID NO: 2;

amino acid sequences which are at least about 50% identical to

the amino acid sequence shown in SEQ ID NO: 7, and

the amino acid sequence shown in SEQ ID NO: 7.

b) a polynucleotide comprising the sequence of SEQ ID NO: 1 or 6.

c) a polynucleotide which hybridizes under stringent conditions to a polynucleotide specified in (a) and (b);

d) a polynucleotide the sequence of which deviates from the polynucleotide sequences specified in (a) to (c) due to the degeneration of the genetic code; and

e) a polynucleotide which represents a fragment, derivative or allelic variation of a polynucleotide sequence specified in (a) to (d).

Furthermore, it has been discovered by the present applicant that a novel serine palmitoyltransferase-like enzyme, particularly a human serine palmitoyltransferase-like enzyme, is a discovery of the present invention. Human serine palmitoyltransferase-like enzyme comprises the amino acid sequence shown in SEQ ID NOS: 2 or 7. Human serine palmitoyltransferase-like enzyme was identified by searching human sequences with the protein having the sequence shown in SEQ ID NO:3 and identified with SwissProt Accession No. 015270 (SEQ ID NO: 3). A coding sequence for human serine palmitoyltransferase-like enzyme is shown in the SEQ ID NOS: 1 or 6. SEQ ID NO: 1 overlaps two ESTs (SEQ ID NOS: 4 and 5) with 100% identity. These ESTs are expressed in HeLaS3, mouse tumors, pancreatic islets, brain, and germinal center B cells.

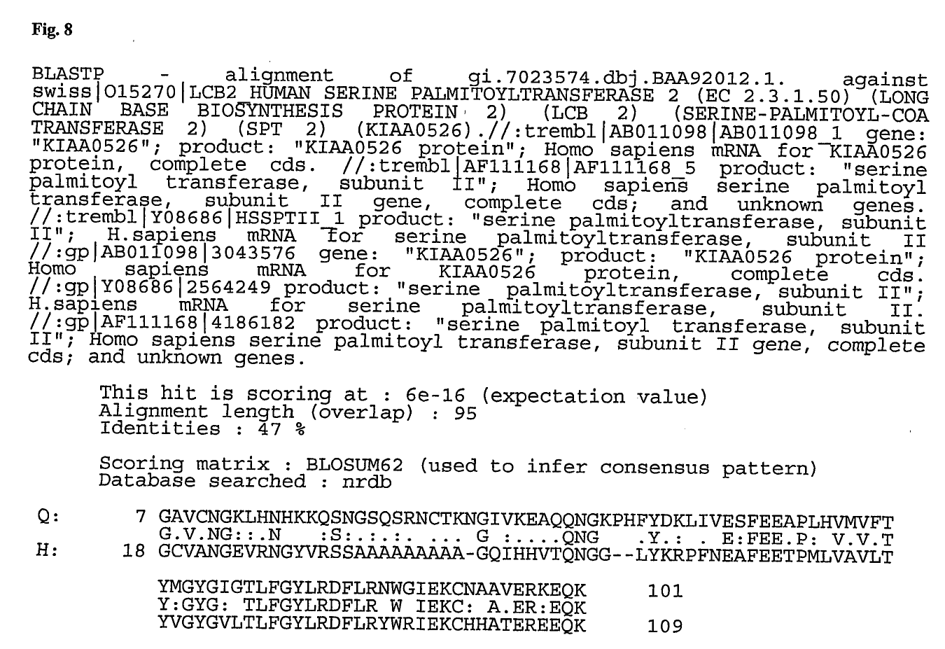

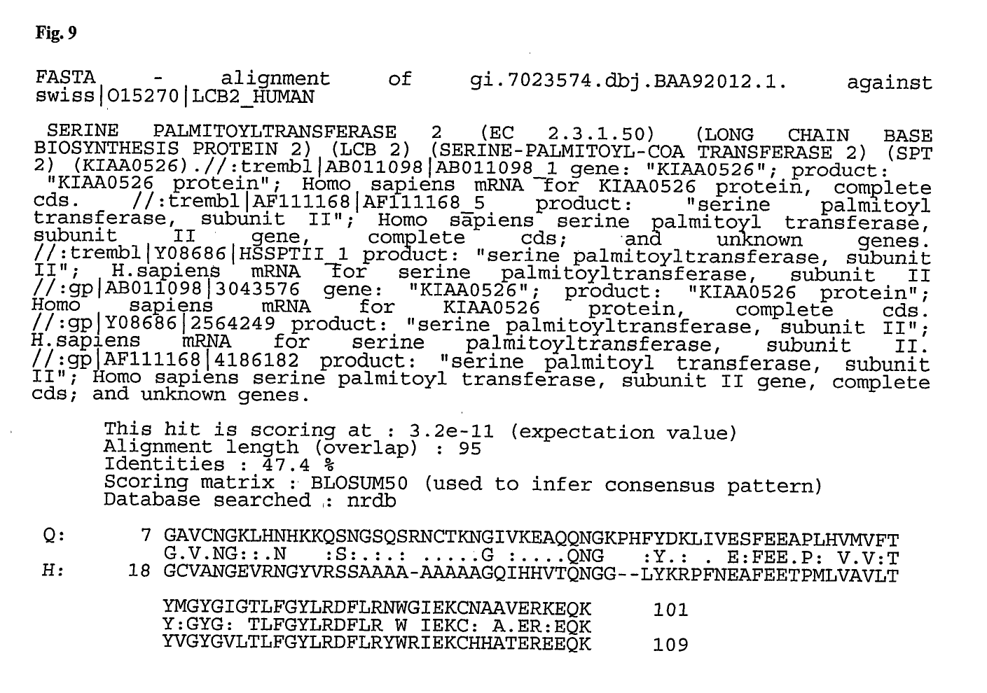

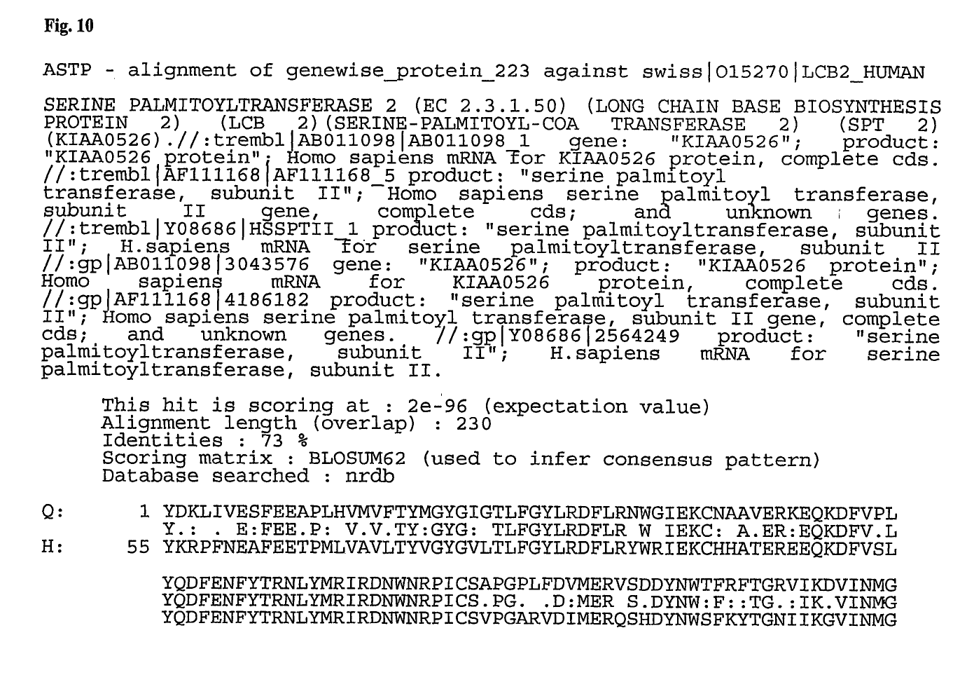

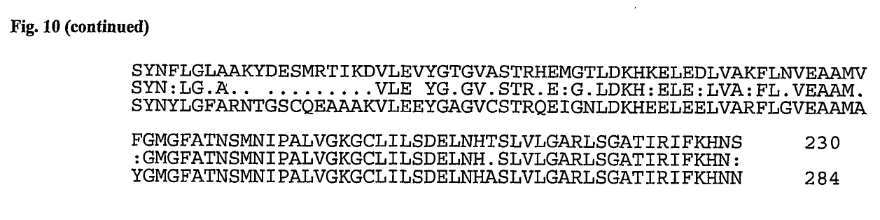

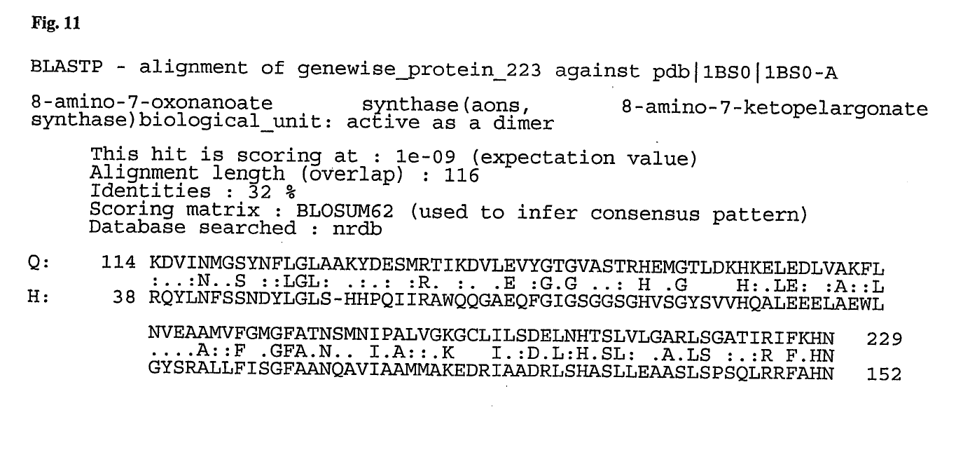

Human serine palmitoyltransferase-like enzyme is 47% identical over 95 amino acids to the human protein identified with SwissProt Accession No. 015270 and annotated as “SERINE PALMITOYLTRANSFERASE 2 (EC 2.3.1.50)” (FIG. 8). Human serine palmitoyltransferase-like enzyme contains a transmembrane region between amino acids 56 and 86.

Human serine palmitoyltransferase-like enzyme of the invention is expected to be useful for the same purposes as previously identified serine palmitoyltransferase enzymes. Thus, human serine palmitoyltransferase-like enzyme can be used in therapeutic methods to treat disorders such as cancer, diabetes, and obesity. Human serine palmitoyltransferase-like enzyme also can be used to screen for human serine palmitoyltransferase-like enzyme agonists and antagonists.

Polypeptides

Human serine palmitoyltransferase-like enzyme polypeptides according to the invention comprise at least 6, 10, 15, 20, 25, 50, 75, 100, 125, 150, or 170 contiguous amino acids selected from the amino acid sequence shown in SEQ ID NOS: 2 or 7 or a biologically active variant thereof, as defined below. A serine palmitoyltransferase-like enzyme polypeptide of the invention therefore can be a portion of a serine palmitoyltransferase-like enzyme protein, a full-length serine palmitoyltransferase-like enzyme protein, or a fusion protein comprising all or a portion of a serine palmitoyltransferase-like enzyme protein.

Biological Active Variants

Human serine palmitoyltransferase-like enzyme polypeptide variants which are biologically active, e.g., retain a serine palmitoyltransferase activity, also are serine palmitoyltransferase-like enzyme polypeptides. Preferably, naturally or non-naturally occurring serine palmitoyltransferase-like enzyme polypeptide variants have amino acid sequences which are at least about 50, 55, 60, 65, or 70, preferably about 75, 80, 85, 90, 96, 96, or 98% identical to the amino acid sequence shown in SEQ ID NO: 2 or 7 or a fragment thereof. Percent identity between a putative serine palmitoyltransferase-like enzyme polypeptide variant and an amino acid sequence of SEQ ID NOS: 2 or 7 is determined with the Needleman/Wunsch algorithm (Needleman and Wunsch, J. Mol. Biol. 48; 443-453, 1970) using a Blosum62 matrix with a gap creation penalty of 8 and a gap extension penalty of 2 (S. Henikoff and J. G. Henikoff, Proc. Natl. Acad. Sci. USA 89:10915-10919, 1992).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a serine palmitoyltransferase-like enzyme polypeptide can be found using computer programs well known in the art, such as DNASTAR software. Whether an amino acid change results in a biologically active serine palmitoyltransferase-like enzyme polypeptide can readily be determined by assaying for serine palmitoyltransferase activity, as described for example, in Williams et al., Arch. Biochem. Biophys, 228, 282-91, 1984; Williams et al., Cancer Res. 44, 1918-23, 1984; Weiss & Stoffel, Eur. J. Biochem. 249, 239-47, 1997; Zweerink et al., J. Biol. Chem. 267, 25032-38, 1992; Mandon et al., Eur. J. Biochem. 198, 667-74, 1991; Holleran et al., J. Lipid Res. 31, 1655-61, 1990; Hanada et al., FEBS Lett. 474, 63-65, 2000; Sandvig et al., Mol. Biol. Cell 7, 1391-404, 1996; Hanada et al., Biochem. Pharmacol. 59, 1211-16, 2000. See also Dickson et al., Methods Enzymol. 311, 3-9, 2000.

Fusion Proteins

Fusion proteins are useful for generating antibodies against serine palmitoyltransferase-like enzyme polypeptide amino acid sequences and for use in various assay systems. For example, fusion proteins can be used to identify proteins which interact with portions of a serine palmitoyltransferase-like enzyme polypeptide. Protein affinity chromatography or library-based assays for protein-protein interactions, such as the yeast two-hybrid or phage display systems, can be used for this purpose. Such methods are well known in the art and also can be used as drug screens.

A serine palmitoyltransferase-like enzyme polypeptide fusion protein comprises two polypeptide segments fused together by means of a peptide bond. The first polypeptide segment comprises at least 6, 10, 15, 20, 25, 50, 75, 100, 125, 150, or 170 contiguous amino acids of SEQ ID NO: 2 or 7 or of a biologically active variant, such as those described above. The first polypeptide segment also can comprise full-length serine palmitoyltransferase-like enzyme protein.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (13FP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the serine palmitoyltransferase-like enzyme polypeptide-encoding sequence and the heterologous protein sequence, so that the serine palmitoyltransferase-like enzyme polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from the complement of SEQ ID NOS: 1 or 6 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human serine palmitoyltransferase-like enzyme polypeptide can be obtained using serine palmitoyltransferase-like enzyme polypeptide polynucleotides (described below) to make suitable probes or primers for screening cDNA expression libraries from other species, such as mice, monkeys, or yeast, identifying cDNAs which encode homologs of serine palmitoyltransferase-like enzyme polypeptide, and expressing the cDNAs as is known in the art.

Polynucleotides

A serine palmitoyltransferase-like enzyme polynucleotide can be single- or double-stranded and comprises a coding sequence or the complement of a coding sequence for a serine palmitoyltransferase-like enzyme polypeptide. A coding sequence for human serine palmitoyltransferase-like enzyme is shown in the SEQ ID NOS: 1 and 6.

Degenerate nucleotide sequences encoding human serine palmitoyltransferase-like enzyme polypeptides, as well as homologous nucleotide sequences which are at least about 50, 55, 60, 65, 70, preferably about 75, 90, 96, or 98% identical to the nucleotide sequence shown in the SEQ ID NOS: 1 or 6 or its complement also are serine palmitoyltransferase-like enzyme polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of serine palmitoyltransferase-like enzyme polynucleotides which encode biologically active serine palmitoyltransferase-like enzyme polypeptides also are serine palmitoyltransferase-like enzyme polynucleotides.

Identification of Polynucleotide Variants and Homologs

Variants and homologs of the serine palmitoyltransferase-like enzyme polynucleotides described above also are serine palmitoyltransferase-like enzyme polynucleotides. Typically, homologous serine palmitoyltransferase-like enzyme polynucleotide sequences can be identified by hybridization of candidate polynucleotides to known serine palmitoyltransferase-like enzyme polynucleotides under stringent conditions, as is known in the art. For example, using the following wash conditions-2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the serine palmitoyltransferase-like enzyme polynucleotides disclosed herein also can be identified by making suitable probes or primers and screening cDNA expression libraries from other species, such as mice, monkeys, or yeast. Human variants of serine palmitoyltransferase-like enzyme polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human serine palmitoyltransferase-like enzyme polynucleotides or serine palmitoyltransferase-like enzyme polynucleotides of other species can therefore be identified by hybridizing a putative homologous serine palmitoyltransferase-like enzyme polynucleotide with a polynucleotide having a nucleotide sequence of SEQ ID NO: 1 or 6 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to serine palmitoyltransferase-like enzyme polynucleotides or their complements following stringent hybridization and/or wash conditions also are serine palmitoyltransferase-like enzyme polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a serine palmitoyltransferase-like enzyme polynucleotide having a nucleotide sequence shown in SEQ ID NO: 1 or 6 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na^{+])+}0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

A serine palmitoyltransferase-like enzyme polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated serine palmitoyltransferase-like enzyme polynucleotides. For example, restriction enzymes and probes can be used to isolate polynucleotide fragments which comprises serine palmitoyltransferase-like nucleotide sequences. Isolated polynucleotides are in preparations which are free or at least 70, 80, or 90% free of other molecules.

Human serine palmitoyltransferase-like enzyme cDNA molecules can be made with standard molecular biology techniques, using serine palmitoyltransferase-like enzyme mRNA as a template. Human serine palmitoyltransferase-like enzyme cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesizes serine palmitoyltransferase-like enzyme polynucleotides. The degeneracy of the genetic code allows alternate nucleotide sequences to be synthesized which will encode a serine palmitoyltransferase-like enzyme polypeptide having, for example, an amino acid sequence shown in SEQ ID NO: 2 or 7 or a biologically active variant thereof.

Extending Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perlin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining Polypeptides

Human serine palmitoyltransferase-like enzyme polypeptides can be obtained, for example, by purification from human cells, by expression of serine palmitoyltransferase-like enzyme polynucleotides, or by direct chemical synthesis.

Protein Purification

Human serine palmitoyltransferase-like enzyme polypeptides can be purified from any cell which expresses the enzyme, including host cells which have been transfected with serine palmitoyltransferase-like enzyme expression constructs. A purified serine palmitoyltransferase-like enzyme polypeptide is separated from other compounds which normally associate with the serine palmitoyltransferase-like enzyme polypeptide in the cell, such as certain proteins, carbohydrates, or lipids, using methods well-known in the art Such methods include, but are not limited to, size exclusion chromatography, ammonium sulfate fractionation, ion exchange chromatography, affinity chromatography, and preparative gel electrophoresis. A preparation of purified serine palmitoyltransferase-like enzyme polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a serine palmitoyltransferase-like enzyme polynucleotide, the polynucleotide can be inserted into an expression vector which contains the necessary elements for the transcription and translation of the inserted coding sequence. Methods which are well known to those skilled in the art can be used to construct expression vectors containing sequences encoding serine palmitoyltransferase-like enzyme polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a serine palmitoyltransferase-like enzyme polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a serine palmitoyltransferase-like enzyme polypeptide, vectors based on SV40 or EBV can be used with an appropriate selectable marker.

Bacterial and Yeast Expression Systems

In bacterial systems, a number of expression vectors can be selected depending upon the use intended for the serine palmitoyltransferase-like enzyme polypeptide. For example, when a large quantity of a serine palmitoyltransferase-like enzyme polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the serine palmitoyltransferase-like enzyme polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding serine palmitoyltransferase-like enzyme polypeptides can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Cornzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in MCGRAW HILL YEARBOOK OF SCIENCE AND TECHNOLOGY, McGraw Hill, New York N.Y., pp. 191-196, 1992).

An insect system also can be used to express a serine palmitoyltransferase-like enzyme polypeptide. For example, in one such system Autographa californica nuclear polyhedrosis virus (AcNPV) is used as a vector to express foreign genes in Spodoptera frugiperda cells or in Trichoplusia larvae. Sequences encoding serine palmitoyltransferase-like enzyme polypeptides can be cloned into a non-essential region of the virus, such as the polyhedrin gene, and placed under control of the polyhedrin promoter. Successful insertion of serine palmitoyltransferase-like enzyme polypeptides will render the polyhedrin gene inactive and produce recombinant virus lacking coat protein. The recombinant viruses can then be used to infect S. frugiperda cells or Trichoplusia larvae in which serine palmitoyltransferase-like enzyme polypeptides can be expressed (Engelhard et al., Proc. Nat. Acad. Sci. 91, 3224-3227, 1994).

Mammalian Expression Systems

A number of viral-based expression systems can be used to express serine palmitoyltransferase-like enzyme polypeptides in mammalian host cells. For example, if an adenovirus is used as an expression vector, sequences encoding serine palmitoyltransferase-like enzyme polypeptides can be ligated into an adenovirus transcription/translation complex comprising the late promoter and tripartite leader sequence. Insertion in a non-essential E1 or E3 region of the viral genome can be used to obtain a viable virus which is capable of expressing a serine palmitoyltransferase-like enzyme polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding serine palmitoyltransferase-like enzyme polypeptides. Such signals include the ATG initiation codon and adjacent sequences. In cases where sequences encoding a serine palmitoyltransferase-like enzyme polypeptide, its initiation codon, and upstream sequences are inserted into the appropriate expression vector, no additional transcriptional or translational control signals may be needed. However, in cases where only coding sequence, or a fragment thereof is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed serine palmitoyltransferase-like enzyme polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express serine palmitoyltransferase-like enzyme polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced serine palmitoyltransferase-like enzyme sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines.

These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprt⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 114, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al, Methods Mol. Biol. 55, 121-131, 1995).

Detecting Expression

Although the presence of marker gene expression suggests that the serine palmitoyltransferase-like enzyme polynucleotide is also present, its presence and expression may need to be confirmed. For example, if a sequence encoding a serine palmitoyltransferase-like enzyme polypeptide is inserted within a marker gene sequence, transformed cells containing sequences which encode a serine palmitoyltransferase-like enzyme polypeptide can be identified by the absence of marker gene function. Alternatively, a marker gene can be placed in tandem with a sequence encoding a serine palmitoyltransferase-like enzyme polypeptide under the control of a single promoter. Expression of the marker gene in response to induction or selection usually indicates expression of the serine palmitoyltransferase-like enzyme polynucleotide.

Alternatively, host cells which contain a serine palmitoyltransferase-like enzyme polynucleotide and which express a serine palmitoyltransferase-like enzyme polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding a serine palmitoyltransferase-like enzyme polypeptide can be detected by DNA-DNA or DNA-RNA hybridization or amplification using probes or fragments or fragments of polynucleotides encoding a serine palmitoyltransferase-like enzyme polypeptide. Nucleic acid amplification-based assays involve the use of oligonucleotides selected from sequences encoding a serine palmitoyltransferase-like enzyme polypeptide to detect transformants which contain a serine palmitoyltransferase-like enzyme polynucleotide.

A variety of protocols for detecting and measuring the expression of a serine palmitoyltransferase-like enzyme polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a serine palmitoyltransferase-like enzyme polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding serine palmitoyltransferase-like enzyme polypeptides include oligolabeling, nick translation, end-labeling, or PCR amplification using a labeled nucleotide. Alternatively, sequences encoding a serine palmitoyltransferase-like enzyme polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding a serine palmitoyltransferase-like enzyme polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode serine palmitoyltransferase-like enzyme polypeptides can be designed to contain signal sequences which direct secretion of soluble serine palmitoyltransferase-like enzyme polypeptides through a prokaryotic or eukaryotic cell membrane or which direct the membrane insertion of membrane-bound serine palmitoyltransferase-like enzyme polypeptide.

As discussed above, other constructions can be used to join a sequence encoding a serine palmitoyltransferase-like enzyme polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the serine palmitoyltransferase-like enzyme polypeptide also can be used to facilitate purification. One such expression vector provides for expression of a fusion protein containing a serine palmitoyltransferase-like enzyme polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif. 3, 263-281, 1992), while the enterokinase cleavage site provides a means for purifying the serine palmitoyltransferase-like enzyme polypeptide from the fusion protein. Vectors which contain fusion proteins are disclosed in Kroll et al., DNA Cell Biol. 12, 441-453, 1993.

Chemical Synthesis

Sequences encoding a serine palmitoyltransferase-like enzyme polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, a serine palmitoyltransferase-like enzyme polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of serine palmitoyltransferase-like enzyme polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, W H Freeman and Co., New York, N.Y., 1983). The composition of a synthetic serine palmitoyltransferase-like enzyme polypeptide can be confirmed by amino acid analysis or sequencing (e.g., the Edman degradation procedure; see Creighton, supra). Additionally, any portion of the amino acid sequence of the serine palmitoyltransferase-like enzyme polypeptide can be altered during direct synthesis and/or combined using chemical methods with sequences from other proteins to produce a variant polypeptide or a fusion protein.

Production of Altered Polypeptides

As will be understood by those of skill in the art, it may be advantageous to produce serine palmitoyltransferase-like enzyme polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter serine palmitoyltransferase-like enzyme polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a serine palmitoyltransferase-like enzyme polypeptide. “Antibody” as used herein includes intact immunoglobulin molecules, as well as fragments thereof, such as Fab, F(ab′)₂, and Fv, which are capable of binding an epitope of a serine palmitoyltransferase-like enzyme polypeptide. Typically, at least 6, 8, 10, or 12 contiguous amino acids are required to form an epitope. However, epitopes which involve non-contiguous amino acids may require more, e.g., at least 15, 25, or SO amino acids.

An antibody which specifically binds to an epitope of a serine palmitoyltransferase-like enzyme polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a serine palmitoyltransferase-like enzyme polypeptide provides a detection signal at least 5-, 10-, or 20-fold higher than a detection signal provided with other proteins when used in an immunochemical assay. Preferably, antibodies which specifically bind to serine palmitoyltransferase-like polypeptides do not detect other proteins in immunochemical assays and can immunoprecipitate a serine palmitoyltransferase-like enzyme polypeptide from solution.

Human serine palmitoyltransferase-like enzyme polypeptides can be used to immunize a mammal, such as a mouse, rat, rabbit, guinea pig, monkey, or human, to produce polyclonal antibodies. If desired, a serine palmitoyltransferase-like enzyme polypeptide can be conjugated to a carrier protein, such as bovine serum albumin, thyroglobulin, and keyhole limpet hemocyanin Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a serine palmitoyltransferase-like enzyme polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 3142, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a serine palmitoyltransferase-like enzyme polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to serine palmitoyltransferase-like enzyme polypeptides. Antibodies with related specificity, but of distinct idiotypic composition, can be generated by chain shuffling from random combinatorial immunoglobin libraries Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 8191).

Antibodies which specifically bind to serine palmitoyltransferase-like enzyme polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which a serine palmitoyltransferase-like enzyme polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of serine palmitoyltransferase-like enzyme gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of serine palmitoyltransferase-like enzyme gene expression can be obtained by designing antisense oligonucleotides which will form duplexes to the control, 5′, or regulatory regions of the serine palmitoyltransferase-like enzyme gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a serine palmitoyltransferase-like enzyme polynucleotide. Antisense oligonucleotides which comprise, for example, 2, 3, 4, or 5 or more stretches of contiguous nucleotides which are precisely complementary to a serine palmitoyltransferase-like enzyme polynucleotide, each separated by a stretch of contiguous nucleotides which are not complementary to adjacent serine palmitoyltransferase-like enzyme nucleotides, can provide sufficient targeting specificity for serine palmitoyltransferase-like enzyme mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular serine palmitoyltransferase-like enzyme polynucleotide sequence.

Antisense oligonucleotides can be modified without affecting their ability to hybridize to a serine palmitoyltransferase-like enzyme polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a serine palmitoyltransferase-like enzyme polynucleotide can be used to generate ribozymes which will specifically bind to mRNA transcribed from the serine palmitoyltransferase-like enzyme polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a serine palmitoyltransferase-like enzyme RNA target can be identified by scanning the target molecule for-ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate serine palmitoyltransferase-like enzyme RNA targets also can be evaluated by testing accessibility to hybridization with complementary oligonucleotides using ribonuclease protection assays. Longer complementary sequences can be used to increase the affinity of the hybridization sequence for the target The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease serine palmitoyltransferase-like enzyme expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Differentially Expressed Genes

Described herein are methods for the identification of genes whose products interact with human serine palmitoyltransferase-like enzyme. Such genes may represent genes which are differentially expressed in disorders including, but not limited to, cancer, diabetes, and obesity. Further, such genes may represent genes which are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, the human serine palmitoyltransferase-like gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

Identification of Differentially Expressed Genes

To identify differentially expressed genes total RNA or, preferably, mRNA is isolated from tissues of interest. For example, RNA samples are obtained from tissues of experimental subjects and from corresponding tissues of control subjects. Any RNA isolation technique which does not select against the isolation of mRNA may be utilized for the purification of such RNA samples. See, for example, Ausubel et al., ed., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples which represent RNA produced by differentially expressed genes are identified by methods well known to those of skill in the art. They include, for example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad. Sci. U.S.A. 88, 2825, 1984), and, preferably, differential display (Liang & Pardee, Science 257, 967-71, 1992; U.S. Pat. No. 5,262,311).

The differential expression information may itself suggest relevant methods for the treatment of disorders involving the human serine palmitoyltransferase-like enzyme. For example, treatment may include a modulation of expression of the differentially expressed genes and/or the gene encoding the human serine palmitoyltransferase-like enzyme. The differential expression information may indicate whether the expression or activity of the differentially expressed gene or gene product or the human serine palmitoyltransferase-like gene or gene product are up-regulated or down-regulated.

Screening Methods

The invention provides assays for screening test compounds which bind to or modulate the activity of a serine palmitoyltransferase-like enzyme polypeptide or a serine palmitoyltransferase-like enzyme polynucleotide. A test compound preferably binds to a serine palmitoyltransferase-like enzyme polypeptide or polynucleotide. More preferably, a test compound decreases or increases serine palmitoyltransferase-like biological activity by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to serine palmitoyltransferase-like enzyme polypeptides or polynucleotides or to affect serine palmitoyltransferase-like enzyme activity or serine palmitoyltransferase-like enzyme gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies, for example, the ATP/GTP binding site of the enzyme or the active site of the serine palmitoyltransferase-like enzyme polypeptide, such that normal biological activity is prevented. Examples of such small molecules include, but are not limited to, small peptides or peptide-like molecules.

In binding assays, either the test compound or the serine palmitoyltransferase-like enzyme polypeptide can comprise a detectable label, such as a fluorescent, radioisotopic, chemiluminescent, or enzymatic label, such as horseradish peroxidase, alkaline phosphatase, or luciferase. Detection of a test compound which is bound to the serine palmitoyltransferase-like enzyme polypeptide can then be accomplished, for example, by direct counting of radioemmission, by scintillation counting, or by determining conversion of an appropriate substrate to a detectable product.

Alternatively, binding of a test compound to a serine palmitoyltransferase-like enzyme polypeptide can be determined without labeling either of the interactants. For example, a microphysiometer can be used to detect binding of a test compound with a serine palmitoyltransferase-like enzyme polypeptide. A microphysiometer (e.g., Cytosensor™ is an analytical instrument that measures the rate at which a cell acidifies its environment using a light-addressable potentiometric sensor (LAPS). Changes in this acidification rate can be used as an indicator of the interaction between a test compound and a serine palmitoyltransferase-like enzyme polypeptide (McConnell et al., Science 257, 1906-1912, 1992).

Determining the ability of a test compound to bind to a serine palmitoyltransferase-like enzyme polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a serine palmitoyltransferase-like enzyme polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300), to identify other proteins which bind to or interact with the serine palmitoyltransferase-like enzyme polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a serine palmitoyltransferase-like enzyme polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the serine palmitoyltransferase-like enzyme polypeptide.

It may be desirable to immobilize either the serine palmitoyltransferase-like enzyme polypeptide (or polynucleotide) or the test compound to facilitate separation of bound from unbound forms of one or both of the interactants, as well as to accommodate automation of the assay. Thus, either the serine palmitoyltransferase-like enzyme polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the enzyme polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a v enzyme polypeptide (or polynucleotide) can be accomplished in any vessel suitable for containing the reactants. Examples of such vessels include microtiter plates, test tubes, and microcentrifuge tubes.

In one embodiment, the serine palmitoyltransferase-like enzyme polypeptide is a fusion protein comprising a domain that allows the serine palmitoyltransferase-like enzyme polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed serine palmitoyltransferase-like enzyme polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a serine palmitoyltransferase-like enzyme polypeptide (or polynucleotide) or a test compound can be immobilized utilizing conjugation of biotin and streptavidin. Biotinylated serine palmitoyltransferase-like enzyme polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a serine palmitoyltransferase-like enzyme polypeptide, polynucleotide, or a test compound, but which do not interfere with a desired binding site, such as the ATP/GTP binding site or the active site of the serine palmitoyltransferase-like enzyme polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the serine palmitoyltransferase-like enzyme polypeptide or test compound, enzyme-linked assays which rely on detecting an activity of the serine palmitoyltransferase-like enzyme polypeptide, and SDS gel electrophoresis under non-reducing conditions.

Screening for test compounds which bind to a serine palmitoyltransferase-like enzyme polypeptide or polynucleotide also can be carried out in an intact cell. Any cell which comprises a serine palmitoyltransferase-like enzyme polypeptide or polynucleotide can be used in a cell-based assay system. A serine palmitoyltransferase-like enzyme polynucleotide can be naturally occurring in the cell or can be introduced using techniques such as those described above. Binding of the test compound to a serine palmitoyltransferase-like enzyme polypeptide or polynucleotide is determined as described above.

Enzyme Assays

Test compounds can be tested for the ability to increase or decrease the serine palmitoyltransferase activity of a human serine palmitoyltransferase-like enzyme polypeptide. Enzyme activity can be measured, for example, as described in Williams et al., Arch. Biochem. Biophys, 228, 282-91, 1984; Williams et al., Cancer Res. 44, 1918-23, 1984; Weiss & Stoffel, Eur. J. Biochem. 249, 239-47, 1997; Zweerink et al., J. Biol. Chem. 267, 25032-38, 1992; Mandon et al., Eur. J. Biochem. 198, 667-74, 1991; Holleran et al., J. Lipid Res. 31, 1655-61, 1990; Hanada et al., FEBS Lett. 474, 63-65, 2000; Sandvig et al., Mol. Biol. Cell 7, 1391-404, 1996; Hanada et al., Biochem. Pharmacol. 59, 1211-16, 2000. See also Dickson et al., Methods Enzymol. 311, 3-9, 2000.

Enzyme assays can be carried out after contacting either a purified serine palmitoyltransferase-like enzyme polypeptide, a cell membrane preparation, or an intact cell with a test compound. A test compound which decreases enzymatic activity of a serine palmitoyltransferase-like enzyme polypeptide by at least about 10, preferably about 50, more preferably about 75, 90, or 100% is identified as a potential therapeutic agent for decreasing serine palmitoyltransferase-like enzyme activity. A test compound which increases enzymatic activity of a human serine palmitoyltransferase-like enzyme polypeptide by at least about 10, preferably about 50, more preferably about 75, 90, or 100% is identified as a potential therapeutic agent for increasing human serine palmitoyltransferase-like enzyme activity.

Gene Expression

In another embodiment, test compounds which increase or decrease serine palmitoyltransferase-like enzyme gene expression are identified. A serine palmitoyltransferase-like enzyme polynucleotide is contacted with a test compound, and the expression of an RNA or polypeptide product of the polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of mRNA or polypeptide expression in the cells can be determined by methods well known in the art for detecting mRNA or polypeptide. Either qualitative or quantitative methods can be used. The presence of polypeptide products of a serine palmitoyltransferase-like enzyme polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a serine palmitoyltransferase-like enzyme polypeptide.

Such screening can be carried out either in a cell-free assay system or in an intact cell. Any cell which expresses a serine palmitoyltransferase-like enzyme polynucleotide can be used in a cell-based assay system. The serine palmitoyltransferase-like enzyme polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions which can be administered to a patient to achieve a therapeutic effect. Pharmaceutical compositions of the invention can comprise, for example, a serine palmitoyltransferase-like enzyme polypeptide, serine palmitoyltransferase-like enzyme polynucleotide, ribozymes or antisense oligonucleotides, antibodies which specifically bind to a serine palmitoyltransferase-like enzyme polypeptide, or mimetics, agonists, antagonists, or inhibitors of a serine palmitoyltransferase-like enzyme polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON's PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

Human serine palmitoyltransferase-like enzyme can be regulated to treat cancer. Cancer is a disease fundamentally caused by oncogenic cellular transformation. There are several hallmarks of transformed cells that distinguish them from their normal counterparts and underlie the pathophysiology of cancer. These include uncontrolled cellular proliferation, unresponsiveness to normal death-inducing signals (immortalization), increased cellular motility and invasiveness, increased ability to recruit blood supply through induction of new blood vessel formation (angiogenesis), genetic instability, and dysregulated gene expression. Various combinations of these aberrant physiologies, along with the acquisition of drug-resistance frequently lead to an intractable disease state in which organ failure and patient death ultimately ensue.

Most standard cancer therapies target cellular proliferation and rely on the differential proliferative capacities between transformed and normal cells for their efficacy. This approach is hindered by the facts that several important normal cell types are also highly proliferative and that cancer cells frequently become resistant to these agents. Thus, the therapeutic indices for traditional anti-cancer therapies rarely exceed 2.0.

The advent of genomics-driven molecular target identification has opened up the possibility of identifying new cancer-specific targets for therapeutic intervention that will provide safer, more effective treatments for cancer patients. Thus, newly discovered tumor-associated genes and their products can be tested for their role(s) in disease and used as tools to discover and develop innovative therapies. Genes playing important roles in any of the physiological processes outlined above can be characterized as cancer targets.

Genes or gene fragments identified through genomics can readily be expressed in one or more heterologous expression systems to produce functional recombinant proteins. These proteins are characterized in vitro for their biochemical properties and then used as tools in high-throughput molecular screening programs to identify chemical modulators of their biochemical activities. Agonists and/or antagonists of target protein activity can be identified in this manner and subsequently tested in cellular and in vivo disease models for anti-cancer activity. Optimization of lead compounds with iterative testing in biological models and detailed pharmacokinetic and toxicological analyses form the basis for drug development and subsequent testing in humans.

Human serine palmitoyltransferase-like enzyme can be regulated to treat diabetes. Diabetes mellitus is a common metabolic disorder characterized by an abnormal elevation in blood glucose, alterations in lipids and abnormalities (complications) in the cardiovascular system, eye, kidney and nervous system. Diabetes is divided into two separate diseases: type 1 diabetes (juvenile onset), which results from a loss of cells which make and secrete insulin, and type 2 diabetes (adult onset), which is caused by a defect in insulin secretion and a defect in insulin action.

Type 1 diabetes is initiated by an autoimmune reaction that attacks the insulin secreting cells (beta cells) in the pancreatic islets. Agents that prevent this reaction from occurring or that stop the reaction before destruction of the beta cells has been accomplished are potential therapies for this disease. Other agents that induce beta cell proliferation and regeneration also are potential therapies.

Type II diabetes is the most common of the two diabetic conditions (6% of the population). The defect in insulin secretion is an important cause of the diabetic condition and results from an inability of the beta cell to properly detect and respond to rises in blood glucose levels with insulin release. Therapies that increase the response by the beta cell to glucose would offer an important new treatment for this disease.

The defect in insulin action in Type II diabetic subjects is another target for therapeutic intervention. Agents that increase the activity of the insulin receptor in muscle, liver, and fat will cause a decrease in blood glucose and a normalization of plasma lipids. The receptor activity can be increased by agents that directly stimulate the receptor or that increase the intracellular signals from the receptor. Other therapies can directly activate the cellular end process, i.e. glucose transport or various enzyme systems, to generate an insulin-like effect and therefore a produce beneficial outcome. Because overweight subjects have a greater susceptibility to Type II diabetes, any agent that reduces body weight is a possible therapy.

Both Type I and Type diabetes can be treated with agents that mimic insulin action or that treat diabetic complications by reducing blood glucose levels. Likewise, agents that reduces new blood vessel growth can be used to treat the eye complications that develop in both diseases.

Human serine palmitoyltransferase-like enzyme can be regulated to treat obesity. Obesity and overweight are defined as an excess of body fat relative to lean body mass. An increase in caloric intake or a decrease in energy expenditure or both can bring about this imbalance leading to surplus energy being stored as fat. Obesity is associated with important medical morbidities and an increase in mortality. The causes of obesity are poorly understood and may be due to genetic factors, environmental factors or a combination of the two to cause a positive energy balance. In contrast, anorexia and cachexia are characterized by an imbalance in energy intake versus energy expenditure leading to a negative energy balance and weight loss. Agents that either increase energy expenditure and/or decrease energy intake, absorption or storage would be useful for treating obesity, overweight, and associated comorbidities. Agents that either increase energy intake and/or decrease energy expenditure or increase the amount of lean tissue would be useful for treating cachexia, anorexia and wasting disorders.

This gene, translated proteins and agents which modulate this gene or portions of the gene or its products are useful for treating obesity, overweight, anorexia, cachexia, wasting disorders, appetite suppression, appetite enhancement, increases or decreases in satiety, modulation of body weight, and/or other eating disorders such as bulimia Also this gene, translated proteins and agents which modulate this gene or portions of the gene or its products are useful for treating obesity/overweight-associated comorbidities including hypertension, type 2 diabetes, coronary artery disease, hyperlipidemia, stroke, gallbladder disease, gout, osteoarthritis, sleep apnea and respiratory problems, some types of cancer including endometrial, breast, prostate, and colon cancer, thrombolic disease, polycystic ovarian syndrome, reduced fertility, complications of pregnancy, menstrual irregularities, hirsutism, stress incontinence, and depression.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a serine palmitoyltransferase-like enzyme polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects serine palmitoyltransferase-like enzyme activity can be administered to a human cell, either in vitro or in vivo, to reduce serine palmitoyltransferase-like enzyme activity. The reagent preferably binds to an expression product of a human serine palmitoyltransferase-like enzyme gene. If the expression product is a protein, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain; lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 106 cells, more preferably about 1.0 μg of DNA per 16 nmole of liposome delivered to about 106 cells, and even more preferably about 2.0 μg of DNA per 16 nmol of liposome delivered to about 106 cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient which increases or decreases serine palmitoyltransferase-like enzyme activity relative to the serine palmitoyltransferase-like enzyme activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a serine palmitoyltransferase-like enzyme gene or the activity of a serine palmitoyltransferase-like enzyme polypeptide by at least about 10, preferably about 50, more preferably about 75, 90, or 100% relative to the absence of the reagent. The effectiveness of the mechanism chosen to decrease the level of expression of a serine palmitoyltransferase-like enzyme gene or the activity of a serine palmitoyltransferase-like enzyme polypeptide can be assessed using methods well known in the art, such as hybridization of nucleotide probes to serine palmitoyltransferase-like enzyme-specific mRNA, quantitative RT-PCR, immunologic detection of a serine palmitoyltransferase-like enzyme polypeptide, or measurement of serine palmitoyltransferase-like enzyme activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Human serine palmitoyltransferase-like enzyme also can be used in diagnostic assays for detecting diseases and abnormalities or susceptibility to diseases and abnormalities related to the presence of mutations in the nucleic acid sequences which encode the enzyme. For example, differences can be determined between the cDNA or genomic sequence encoding serine palmitoyltransferase-like enzyme in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced-when combined with PCR For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of a serine palmitoyltransferase-like enzyme also can be detected in various tissues. Assays used to detect levels of the receptor polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention

### EXAMPLE 1

Detection of Serine Palmitoyltransferase-Like Enzyme Activity

The polynucleotide of SEQ ID NO: 1 or 6 is inserted into the expression vector pCEV4 and the expression vector pCEV4-serine palmitoyltransferase-like enzyme polypeptide obtained is transfected into human embryonic kidney 293 cells. From these cells extracts are obtained and SPT activity is assayed in a 300-μl volume containing 50 mM HEPES, 8.3, 50 mM pyridoxal phosphate, 2 mM serine (20 μCi/ml) 0.2 mM palmitoyl CoA, 1 mM NADPH, 2.4 mM glucose-6-phosphate, and 10 units of glucose-6-phosphate dehydrogenase. The reaction is initiated by adding 0.5-1 mg of the cell extract, and after 10 min at 37° C., it is terminated by the addition of 100 μl of 2 M NH₄OH and 0.75 ml of CHCl₃:MeOH (1:2). After vortexing, an additional 0.75 ml of CHCl₃:MeOH (1:2), 1 ml of CHCl₃, and 2 ml of 0.5 M NH₄OH are added with vortexing after each addition. After brief centrifugation to separate the phases, the top layer is aspirated off. The organic phase is washed three times (or until clear) with 30 mM KCl, and a fixed volume is dried under N2 and resuspended in scintillation fluid for counting. SPT activity is expressed in pmol of serine converted per mg of the cell extract per min. It is shown that the polypeptide of SEQ ID NO: 2 and 7 has a serine palmitoyltransferase-like enzyme activity.

### EXAMPLE 2

Expression of Recombinant Human Serine Palmitoyltransferase-Like Enzyme

The Pichia pastoris expression vector pPICZB (Invitrogen, San Diego, Calif.) is used to produce large quantities of recombinant human serine palmitoyltransferase-like polypeptides in yeast. The serine palmitoyltransferase-like enzyme-encoding DNA sequence is derived from SEQ ID NO: 1 or 6. Before insertion into vector pPICZB, the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriction enzymes the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, Calif.) according to manufacturer's instructions. Purified human serine palmitoyltransferase-like enzyme polypeptide is obtained.

### EXAMPLE 3

Identification of Test Compounds That Bind to Serine Palmitoyltransferase-Like Enzyme Polypeptides

Purified serine palmitoyltransferase-like enzyme polypeptides comprising a glutathione-S-transferase protein and absorbed onto glutathione-derivatized wells of 96-well microtiter plates are contacted with test compounds from a small molecule library at pH 7.0 in a physiological buffer solution. Human serine palmitoyltransferase-like enzyme polypeptides comprise the amino acid sequence shown in SEQ ID NO: 2 or 7. The test compounds comprise a fluorescent tag. The samples are incubated for 5 minutes to one hour. Control samples are incubated in the absence of a test compound.

The buffer solution containing the test compounds is washed from the wells. Binding of a test compound to a serine palmitoyltransferase-like enzyme polypeptide is detected by fluorescence measurements of the contents of the wells. A test compound which increases the fluorescence in a well by at least 15% relative to fluorescence of a well in which a test compound is not incubated is identified as a compound which binds to a serine palmitoyltransferase-like enzyme polypeptide.

### EXAMPLE 4

Identification of a Test Compound Which Decreases Serine Palmitoyltransferase-Like Enzyme Gene Expression

A test compound is administered to a culture of human cells transfected with a serine palmitoyltransferase-like enzyme expression construct and incubated at 37° C. for 10 to 45 minutes. A culture of the same type of cells which have not been transfected is incubated for the same time without the test compound to provide a negative control.

RNA is isolated from the two cultures as described in Chirgwin et al., Biochem. 18, 5294-99, 1979). Northern blots are prepared using 20 to 30 μg total RNA and hybridized with a ³²P-labeled serine palmitoyltransferase-like enzyme-specific probe at 65° C. in Express-hyb (CLONTECH). The probe comprises at least 11 contiguous nucleotides selected from the complement of SEQ ID NO: 1 ir 6. A test compound which decreases the serine palmitoyltransferase-like enzyme-specific signal relative to the signal obtained in the absence of the test compound is identified as an inhibitor of serine palmitoyltransferase-like enzyme gene expression.

### EXAMPLE 5

Identification of a Test Compound Which Decreases Serine Palmitoyltransferase-Like Enzyme Activity

A test compound is administered to a culture of human cells transfected with a serine palmitoyltransferase-like enzyme expression construct and incubated at 37° C. for 10 to 45 minutes. A culture of the same type of cells which have not been transfected is incubated for the same time without the test compound to provide a negative control. Serine palmitoyltransferase activity is measured as described in Weiss & Stoffel, Eur. J. Biochem. 249, 239-47, 1997.

A test compound which decreases the serine palmitoyltransferase activity of the serine palmitoyltransferase-like enzyme relative to the serine palmitoyltransferase activity in the absence of the test compound is identified as an inhibitor of serine palmitoyltransferase-like enzyme activity.

1. An isolated polynucleotide encoding a serine palmitoyltransferase-like enzyme polypeptide and being selected from the group consisting of:
a) a polynucleotide encoding a serine palmitoyltransferase-like enzyme polypeptide comprising an amino acid sequence selected form the group consisting of:
amino acid sequences which are at least about 50% identical to
the amino acid sequence shown in SEQ ID NO: 2;
the amino acid sequence shown in SEQ ID NO: 2;
amino acid sequences which are at least about 50% identical to
the amino acid sequence shown in SEQ ID NO: 7; and
the amino acid sequence shown in SEQ ID NO: 7.
b) a polynucleotide comprising the sequence of SEQ ID NO: 1 or 6;
c) a polynucleotide which hybridizes under stringent conditions to a polynucleotide specified in (a) and (b);
d) a polynucleotide the sequence of which deviates from the polynucleotide sequences specified in (a) to (c) due to the degeneration of the genetic code; and
e) a polynucleotide which represents a fragment, derivative or allelic variation of a polynucleotide sequence specified in (a to (d).

2. An expression vector containing any polynucleotide of claim 1.

3. A host cell containing the expression vector of claim 2.

4. A substantially purified serine palmitoyltransferase-like enzyme polypeptide encoded by a polynucleotide of claim 1.

5. A method for producing a serine palmitoyltransferase-like enzyme polypeptide, wherein the method comprises the following steps:
a) culturing the host cell of claim 3 under conditions suitable for the expression of the serine palmitoyltransferase-like enzyme polypeptide; and
b) recovering the serine palmitoyltransferase-like enzyme polypeptide from the host cell culture.

6. A method for detection of a polynucleotide encoding a serine palmitoyltransferase-like enzyme polypeptide in a biological sample comprising the following steps:
a) hybridizing any polynucleotide of claim 1 to a nucleic acid material of a biological sample, thereby forming a hybridization complex; and
b) detecting said hybridization complex.

7. The method of claim 6, wherein before hybridization, the nucleic acid material of the biological sample is amplified.

8. A method for the detection of a polynucleotide of claim 1 or a serine palmitoyltransferase-like enzyme polypeptide of claim 4 comprising the steps of:
contacting a biological sample with a reagent which specifically interacts with the polynucleotide or the serine palmitoyltransferase-like enzyme polypeptide.

9. A diagnostic kit for conducting the method of any one of claims 6 to 8.

10. A method of screening for agents which decrease the activity of a serine palmitoyltransferase-like enzyme, comprising the steps of:
contacting a test compound with any serine palmitoyltransferase-like enzyme polypeptide encoded by any polynucleotide of claim 1;
detecting binding of the test compound to the serine palmitoyltransferase-like enzyme polypeptide, wherein a test compound which binds to the polypeptide is identified as a potential therapeutic agent for decreasing the activity of a serine palmitoyltransferase-like enzyme.

11. A method of screening for agents which regulate the activity of a serine palmitoyltransferase-like enzyme, comprising the steps of:
contacting a test compound with a serine palmitoyltransferase-like enzyme polypeptide encoded by any polynucleotide of claim 1; and
detecting a serine palmitoyltransferase-like enzyme activity of the polypeptide, wherein a test compound which increases the serine palmitoyltransferase-like enzyme activity is identified as a potential therapeutic agent for increasing the activity of the serine palmitoyltransferase-like enzyme, and wherein a test compound which decreases the serine palmitoyltransferase-like enzyme activity of the polypeptide is identified as a potential therapeutic agent for decreasing the activity of the serine palmitoyltransferase-like enzyme.

12. A method of screening for agents which decrease the activity of a serine palmitoyltransferase-like enzyme, comprising the steps of:
contacting a test compound with any polynucleotide of claim 1 and detecting binding of the test compound to the polynucleotide, wherein a test compound which binds to the polynucleotide is identified as a potential therapeutic agent for decreasing the activity of serine palmitoyltransferase-like enzyme.

13. A method of reducing the activity of serine palmitoyltransferase-like enzyme, comprising the steps of:
contacting a cell with a reagent which specifically binds to any polynucleotide of claim 1 or any serine palmitoyltransferase-like enzyme polypeptide of claim 4, whereby the activity of serine palmitoyltransferase-like enzyme is reduced.

14. A reagent that modulates the activity of a serine palmitoyltransferase-like enzyme polypeptide or a polynucleotide wherein said reagent is identified by the method of any of the claim 10 to 12.

15. A pharmaceutical composition, comprising:
the expression vector of claim 2 or the reagent of claim 14 and a pharmaceutically acceptable carrier.

16. Use of the pharmaceutical composition of claim 15 for modulating the activity of a serine palmitoyltransferase-like enzyme in a disease.

17. Use of claim 16 wherein the disease is cancer, diabetes or obesity.

18. A cDNA encoding a polypeptide comprising the amino acid sequence shown in SEQ ID NO: 2 or 7.

19. The cDNA of claim 18 which comprises SEQ ID NO: 1 or 6.

20. The cDNA of claim 18 which consists of SEQ ID NO: 1 or 6.

21. An expression vector comprising a polynucleotide which encodes a polypeptide comprising the amino acid sequence shown in SEQ ID NO: 2 or 7.

22. The expression vector of claim 21 wherein the polynucleotide consists of SEQ ED NO: 1 or 6.

23. A host cell comprising an expression vector which encodes a polypeptide comprising the amino acid sequence shown in SEQ ID NO: 2 or 7.

24. The host cell of claim 23 wherein the polynucleotide consists of SEQ ID NO: 1 or 6.

25. A purified polypeptide comprising the amino acid sequence shown in SEQ ID NO: 2 or 7.

26. The purified polypeptide of claim 25 which consists of the amino acid sequence shown in SEQ ID NO: 2 or 7.

27. A fusion protein comprising a polypeptide having the amino acid sequence shown in SEQ ID NO: 2 or 7.

28. A method of producing a polypeptide comprising the amino acid sequence shown in SEQ ID NO: 2 or 7, comprising the steps of:
culturing a host cell comprising an expression vector which encodes the polypeptide under conditions whereby the polypeptide is expressed; and
isolating the polypeptide.

29. The method of claim 28 wherein the expression vector comprises SEQ ID NO: 1 or 6.

30. A method of detecting a coding sequence for a polypeptide comprising the amino acid sequence shown in SEQ ID NO: 2 or 7, comprising the steps of:
hybridizing a polynucleotide comprising 11 contiguous nucleotides of SEQ ID NO: 1 or 6 to nucleic acid material of a biological sample, thereby forming a hybridization complex; and
detecting the hybridization complex.

31. The method of claim 30 further comprising the step of amplifying the nucleic acid material before the step of hybridizing.

32. A kit for detecting a coding sequence for a polypeptide comprising the amino acid sequence shown in SEQ ID NO: 2 or 7, comprising:
a polynucleotide comprising 11 contiguous nucleotides of SEQ ID NO: 1 or 6; and
instructions for the method of claim 30.

33. A method of detecting a polypeptide comprising the amino acid sequence shown in SEQ ID NO: 2 or 7, comprising the steps of:
contacting a biological sample with a reagent that specifically binds to the polypeptide to form a reagent-polypeptide complex; and
detecting the reagent-polypeptide complex.

34. The method of claim 33 wherein the reagent is an antibody.

35. A kit for detecting a polypeptide comprising the amino acid sequence shown in SEQ ID NO: 2 or 7, comprising:
an antibody which specifically binds to the polypeptide; and
instructions for the method of claim 33.

36. A method of screening for agents which can modulate the activity of a human serine palmitoyltransferase-like enzyme, comprising the steps of:
contacting a test compound with a polypeptide comprising an amino acid sequence selected from the group consisting of: (1) amino acid sequences which are at least about 50% identical to the amino acid sequence shown in SEQ ID NO: 2 or 7 and (2) the amino acid sequence shown in SEQ ID NO: 2 or 7; and
detecting binding of the test compound to the polypeptide, wherein a test compound which binds to the polypeptide is identified as a potential agent for regulating activity of the human serine palmitoyltransferase-like enzyme.

37. The method of claim 36 wherein the step of contacting is in a cell.

38. The method of claim 36 wherein the cell is in vitro.

39. The method of claim 36 wherein the step of contacting is in a cell-free system.

40. The method of claim 36 wherein the polypeptide comprises a detectable label.

41. The method of claim 36 wherein the test compound comprises a detectable label.

42. The method of claim 36 wherein the test compound displaces a labeled ligand which is bound to the polypeptide.

43. The method of claim 36 wherein the polypeptide is bound to a solid support.

44. The method of claim 36 wherein the test compound is bound to a solid support.

45. A method of screening for agents which modulate an activity of a human serine palmitoyltransferase-like enzyme, comprising the steps of:
contacting a test compound with a polypeptide comprising an amino acid sequence selected from the group consisting of: (1) amino acid sequences which are at least about 50% identical to the amino acid sequence shown in SEQ ID NO: 2 or 7 and (2) the amino acid sequence shown in SEQ ID NO: 2 or 7; and
detecting an activity of the polypeptide, wherein a test compound which increases the activity of the polypeptide is identified as a potential agent for increasing the activity of the human serine palmitoyltransferase-like enzyme, and wherein a test compound which decreases the activity of the polypeptide is identified as a potential agent for decreasing the activity of the human serine palmitoyltransferase-like enzyme.

46. The method of claim 45 wherein the step of contacting is in a cell.

47. The method of claim 45 wherein the cell is in vitro.

48. The method of claim 45 wherein the step of contacting is in a cell-free system.

49. A method of screening for agents which modulate an activity of a human serine palmitoyltransferase-like enzyme, comprising the steps of:
contacting a test compound with a product encoded by a polynucleotide which comprises the nucleotide sequence shown in SEQ ID NO: 1 or 6; and
detecting binding of the test compound to the product, wherein a test compound which binds to the product is identified as a potential agent for regulating the activity of the human serine palmitoyltransferase-like enzyme.

50. The method of claim 49 wherein the product is a polypeptide.

51. The method of claim 49 wherein the product is RNA.

52. A method of reducing activity of a human serine palmitoyltransferase-like enzyme, comprising the step of:
contacting a cell with a reagent which specifically binds to a product encoded by a polynucleotide comprising the nucleotide sequence shown in SEQ ID NO: 1 or 6, whereby the activity of a human serine palmitoyltransferase-like enzyme is reduced.

53. The method of claim 52 wherein the product is a polypeptide.

54. The method of claim 53 wherein the reagent is an antibody.

55. The method of claim 52 wherein the product is RNA.

56. The method of claim 55 wherein the reagent is an antisense oligonucleotide.

57. The method of claim 56 wherein the reagent is a ribozyme.

58. The method of claim 52 wherein the cell is in vitro.

59. The method of claim 52 wherein the cell is in vivo.

60. A pharmaceutical composition, comprising:
a reagent which specifically binds to a polypeptide comprising the amino acid sequence shown in SEQ ID NO: 2 or 7; and
a pharmaceutically acceptable carrier.

61. The pharmaceutical composition of claim 60 wherein the reagent is an antibody.

62. A pharmaceutical composition, comprising:
a reagent which specifically binds to a product of a polynucleotide comprising the nucleotide sequence shown in SEQ ID NO: 1 or 6; and
a pharmaceutically acceptable carrier.

63. The pharmaceutical composition of claim 62 wherein the reagent is a ribozyme.

64. The pharmaceutical composition of claim 62 wherein the reagent is an antisense oligonucleotide.

65. The pharmaceutical composition of claim 62 wherein the reagent is an antibody.

66. A pharmaceutical composition, comprising:
an expression vector encoding a polypeptide comprising the amino acid sequence shown in SEQ ID NO: 2 or 7; and
a pharmaceutically acceptable carrier.

67. The pharmaceutical composition of claim 66 wherein the expression vector comprises SEQ ID NO: 1 or 6.

68. A method of treating a serine palmitoyltransferase-like enzyme dysfunction related disease, wherein the disease is selected from cancer, diabetes and obesity comprising the step of:
administering to a patient in need thereof a therapeutically effective dose of a reagent that modulates a function of a human serine palmitoyltransferase-like enzyme, whereby symptoms of the serine palmitoyltransferase-like enzyme dysfunction related disease are ameliorated.

69. The method of claim 68 wherein the reagent is identified by the method of claim 36.

70. The method of claim 68 wherein the reagent is identified by the method of claim 45.

71. The method of claim 68 wherein the reagent is identified by the method of claim 49.


SEQUENCE LISTING (7 sequences)

SEQ ID NO: 1 - DNA, 525 bp, Homo sapiens
atggctaaccctggaggtggtgctgtttgcaacgggaaacttcacaatcacaagaaacagagcaatggctcacaaagcagaaactgcacaaagaatggaatagtgaaggaagcccagcaaaatgggaagccacatttttatgataagctcattgttgaatcgtttgaggaagcaccccttcatgttatggttttcacttacatgggatatggaattggaaccctgtttggctatctcagagactttttaagaaactggggaatagaaaaatgcaacgcagctgtggaacgaaaagaacaaaaagtacgtatgcgcacctccctggatctttgtcaatgcctactcctctctaaagtgttctcagaagtggtgatgcaggtgcagattctagaaagcatgaggtgctcaggaactattcagggcaaatttcattcatctccacctgctaaaccccattacccatgggcttatggacctgtttttacaaacatctcatgggcaactactatttgccacataccaaac

SEQ ID NO: 2 - protein, 175 aa, Homo sapiens
MANPGGGAVCNGKLHNHKKQSNGSQSRNCTKNGIVKEAQQNGKPHFYDKLIVESFEEAPLHVMVFTYMGYGIGTLFGYLRDFLRNWGIEKCNAAVERKEQKVRMRTSLDLCQCLLLSKVFSEVVMQVQILESMRCSGTIQGKFHSSPPAKPHYPWAYGPVFTNISWATTICHIPN

SEQ ID NO: 3 - protein, 562 aa, Homo sapiens
MRPEPGGCCCRRTVRANGCVANGEVRNGYVRSSAAAAAAAAAGQIHHVTQNGGLYKRPFNEAFEETPMLVAVLTYVGYGVLTLFGYLRDFLRYWRIEKCHHATEREEQKDFVSLYQDFENFYTRNLYMRIRDNWNRPICSVPGARVDIMERQSHDYNWSFKYTGNIIKGVINMGSYNYLGFARNTGSCQEAAAKVLEEYGAGVCSTRQEIGNLDKHEELEELVARFLGVEAAMAYGMGFATNSMNIPALVGKGCLILSDELNHASLVLGARLSGATIRIFKHNNMQSLEKLLKDAIVYGQPRTRRPWKKILILVEGIYSMEGSIVRLPEVIALKKKYKAYLYLDEAHSIGALGPTGRGVVEYFGLDPEDVDVMMGTFTKSFGASGGYIGGKKELIDYLRTHSHSAVYATSLSPPVVEQIITSMKCIMGQDGTSLGKECVQQLAENTRYFRRRLKEMGFIIYGNEDSPVVPLMLYMPAKIGAFGREMLKRNIGVVVVGFPATPIIESRARFCLSAAHTKEILDTALKEIDEVGDLLQLKYSRHRLVPLLDRPFDETTYEETED

SEQ ID NO: 4 - DNA, 944 bp, Homo sapiens
  misc_feature (1)...(944): n = A,T,C or G
gttgctantcgtcgcctactactactacaatgctgcgagaagacgacagaagggcagagactttttaagaaactggggaatagaaaaatgcaacgcagctgtggaacgaaaagaacaaaaagtacgtatgcgcacctccctggatctttgtcaatgcctactcctctctaaagtgttctcagaagtggtgatgcaggtgcagattctagaaagcatgaggtgctcaggaactattcagggcaaatttcattcatctccacctgctaaaccccattacccatgggcttatggacctgtttttacaaacatctcatgggcaactactatttgccacataccaaactaaggcttttcccataccttgtaatatttaatttttgtaacaaccccatgatatagatattaaactataattttgaagataaaaacatcaaaatataaaagcaagtgctcctgggtgggactgtactttggtcttctagttgtaagttgggtattatttatgctctatcctgaaaaggaaagtgcatttgagtgaactgggacttttttatttcatctaaatctgatttctcacctttgacactattaatattttgaaccaattttttgggcattgtaagaattttaacagcatctgaaaaatatagcattaaaccactaaagctaaggaccctgatgaagtcactgcaccctccactacctctactggaacaggcactggtatncactgctgagagacccatagacggttcacatcacangactctgtggagacaatccccagtaccacttggacccggtagactcgtgggtggcgaganccanaaanaaacacatcctgcagtcactccaggaagcccatcctaggaaaaggggagagtatacatcaagggaacacccccanggacaaaanaatttgacaacagtctttannccctaac

SEQ ID NO: 5 - DNA, 646 bp, Homo sapiens
  misc_feature (1)...(646): n = A,T,C or G
aactgcacaaagaatggaatagtgaaggaagcccagcaaaatgggaagccacatttttatgataagctcattgttgaatcgtttgaggaagcaccccttcatgttatggttttcacttacatgggatatggaattggaaccctgtttggctatctcagagactttttaagaaactggggaatagaaaaatgcaacgcagctgtggaacgaaaagaacaaaaagattttgtgccactgtatcaagactttgaaaatttttatacaagaaacctttacatgcgaatcagagacaactggaaccggcccatctgcagtgccccagggctctgtttgatgtgatggagagggtatcagacgactataactggacgtttaggtttactggaagagtcatcaaagatgtcatcaacatgggctcctataacttccttggtcttgcagcccagtatgatgagtctatgagggaccataaaggatgttttagaggtgtatgggcacangcgttgccagcaccaggcatgaaatgggtgaattcacctcatcctggcagaatgcctctgtgttggaagggtcttgcnagctgactccgtgccacaggggccacctgggtccttggaaaattggcaggcactcttncccgtttgaa

SEQ ID NO: 6 - DNA, 690 bp, Homo sapiens
tatgataagctcattgttgaatcgtttgaggaagcaccccttcatgttatggttttcacttacatgggatatggaattggaaccctgtttggctatctcagagactttttaagaaactggggaatagaaaaatgcaacgcagctgtggaaagaaaagaacaaaaagattttgtgccactgtatcaagactttgaaaatttttatacaagaaacctttacatgcgaatcagagacaactggaaccggcccatctgcagtgccccagggcctctgtttgatgtgatggagagggtatcggacgactataactggacgtttaggtttactggaagagtcatcaaagatgtcatcaacatgggctcctataacttccttggtcttgcagccaagtatgatgagtctatgaggacaataaaggatgttttagaggtgtatggcacaggcgtggccagcaccaggcatgaaatgggcaccttggataagcacaaggagttggaggaccttgtggctaagttcctgaatgtggaagcagctatggtctttgggatgggattcgcaactaactcaatgaatatcccagcattagttggaaagggatgcctcattttaagtgatgagttaaaccacacatcgcttgtgcttggggcccgactctcaggtgcaaccataagaatcttcaaacacaacagt

SEQ ID NO: 7 - protein, 230 aa, Homo sapiens
YDKLIVESFEEAPLHVMVFTYMGYGIGTLFGYLRDFLRNWGIEKCNAAVERKEQKDFVPLYQDFENFYTRNLYMRIRDNWNRPICSAPGPLFDVMERVSDDYNWTFRFTGRVIKDVINMGSYNFLGLAAKYDESMRTIKDVLEVYGTGVASTRHEMGTLDKHKELEDLVAKFLNVEAAMVFGMGFATNSMNIPALVGKGCLILSDELNHTSLVLGARLSGATIRIFKHNS